(19) United States
(12) Patent Application Publication
(10) Pub. No.: US 2022/0362130 A1
(43) Pub. Date: Nov. 17, 2022

(54) COMPOSITIONS AND METHODS FOR TREATING KERATIN FIBERS

(71) Applicant: L'Oreal, Paris (FR)

(72) Inventors: Stephanie MIDORI LAGA, Rahway, NJ (US); Seyma ASLAN, Clifton, NJ (US)

(21) Appl. No.: 17/245,480

(22) Filed: Apr. 30, 2021

(51) Int. Cl. A61K 8/73; A61Q 5/00

(52) U.S. Cl. CPC A61K 8/738; A61Q 5/00; A61K 2800/5426

(57) ABSTRACT

The disclosure relates to compositions comprising (a) at least one cyclodextrin or derivative thereof; (b) at least one solvent; (c) optionally at least one cationic surfactant; (c) optionally at least one emulsifier; (d) optionally at least one silicone; and (e) optionally at least one oil. The compositions can be used for imparting benefits to keratin fibers, such as elongation of hair curls. The disclosure also relates to methods of using the compositions.

### TECHNICAL FIELD

The present disclosure relates to compositions and methods for treating keratinous fibers, such as curly or wavy hair. The compositions improve the elongation of hair curls or waves while simultaneously delivering benefits such as softness, smoothness, and/or frizz control to the hair. The disclosure also relates to methods of using the compositions.

### BACKGROUND

Curly or wavy hair usually appears shorter than its actual length. In the multicultural beauty market, there is a desire for products that can elongate hair curls and give more visual length to hair. In addition, reducing the curls of very curly hair may increase the manageability and ease of styling of such hair.

Traditional curl-elongation products generally include heavy galenics or oils, which usually leave a greasy or oily feeling. Products that provide hold, such as styling gels, can also give elongating effects, but these products typically flake on hair. Therefore, the use of these elongation products generally requires frequent hair washing, which may be undesirable for consumers who have high curl patterns and prefer limited number of washdays per week. In addition, traditional elongation of hair, including relaxing or straightening hair, usually involves chemical treatment that may cause damage to the hair fibers and/or irritate the scalp.

As such, consumers desire new and improved styling compositions that can deliver visible elongation to hair curls, and at the same time impart various additional advantageous properties to the hair such as good curl definition, curl hold, hair moisture, softness, smoothness, good bounce, good shine, and/or frizz control.

It has now surprisingly been found that compositions comprising at least one cyclodextrin are able to improve and/or maintain the elongation of hair curls, and may provide other benefits such as hair moisture, softness, shine, and/or frizz control to the hair.

### SUMMARY

The present disclosure relates to compositions for treating keratinous fibers, such as curly or wavy hair, that may provide beneficial effects, such a curl elongation, to the hair. The compositions are optionally leave-in compositions. The compositions may comprise, in certain embodiments: (a) at least one cyclodextrin or derivative thereof; (b) at least one solvent; (c) optionally at least one cationic surfactant; (c) optionally at least one emulsifier; (d) optionally at least one silicone; and (e) optionally at least one oil. In various embodiments, the at least one solvent comprises water and at least one additional solvent that can improve the solubility of the at least one cyclodextrin or derivative thereof, and the weight ratio of cyclodextrin:additional solvent ranges from about 1.2:0.1 to about 1:1, such as from about 1:0.1 to about 1:0.8, from about 1:0.15 to about 1:0.6, or about 1:0.2 to about 1:0.5. For example, the weight ratio of cyclodextrin:additional solvent may be about 1:0.25. In various embodiments, the at least one additional solvent is chosen from linear or branched, saturated or unsaturated, substituted or unsubstituted C₃-C₈ polyols, such as, for example, ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, or a mixture of any two or more of the foregoing. However, in at least some embodiments, no additional solvent is needed to improve the solubility of the at least one cyclodextrin or derivative thereof, although it may be desirable to include one or more of these solvents as part of the solvent mixture.

In various embodiments, the at least one cyclodextrin may be chosen from those of formula:

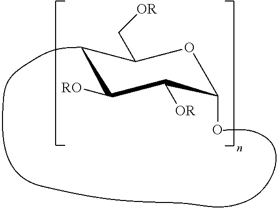

where R is chosen from H, CH₃, or a hydroxypropyl group, and n ranges from 6-8. For example, the at least one cyclodextrin or derivative thereof may be α-cyclodextrin, β-cyclodextrin, γ-cyclodextrin, a methyl derivative of α-cyclodextrin, β-cyclodextrin, or γ-cyclodextrin, a hydroxypropyl derivative of α-cyclodextrin, β-cyclodextrin, or γ-cyclodextrin, or a mixture of any two or more of the foregoing. The at least one cyclodextrin or derivative thereof may be present in an amount ranging from about 0.01% to about 20%, such as from about 0.1% to about 18%, from about 0.5% to about 15%, or from about 1% to about 13% by weight, relative to the total weight of the composition, for example from about 0.5% to about 10%, from about 1% to about 8%, from about 3% to about 7%, or from about 4% to about 6%.

In some embodiments, a composition according to the disclosure comprises from about 0.5% to about 10%, from about 1% to about 8%, from about 3% to about 7%, or from about 4% to about 6% by weight of at least one cyclodextrin chosen from α-cyclodextrin, β-cyclodextrin, γ-cyclodextrin, or a mixture of two or more of the foregoing, and a solvent comprising water and at least one additional solvent chosen from linear or branched, saturated or unsaturated, substituted or unsubstituted C₃-C₈ polyols, such as, for example, ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, or a mixture of any two or more of the foregoing. The weight ratio of cyclodextrin:additional solvent may range from about 1:0.15 to about 1:0.6, or about 1:0.2 to about 1:0.5, and in at least one embodiment is about 1:0.25.

In an embodiment, a composition according to the disclosure comprises at least one cyclodextrin chosen from α-cyclodextrin, β-cyclodextrin, γ-cyclodextrin, or a mixture of two or more of the foregoing, and a solvent comprising water and at least one additional solvent chosen from linear or branched, saturated or unsaturated, substituted or unsubstituted C₃-C₈ polyols, for example hexylene glycol, pentylene glycol, or a mixture thereof, where the weight ratio of cyclodextrin:additional solvent ranges from about 1:0.15 to about 1:0.6, or about 1:0.2 to about 1:0.5. In another embodiment, a composition according to the disclosure comprises from about 3% to about 7% by weight of at least one cyclodextrin chosen from α-cyclodextrin, β-cyclodextrin, γ-cyclodextrin, or a mixture of two or more of the foregoing, and a solvent comprising water and at least one additional solvent chosen from linear or branched, saturated or unsaturated, substituted or unsubstituted C₃-C₈ polyols, for example hexylene glycol, pentylene glycol, or a mixture thereof, where the weight ratio of cyclodextrin:additional solvent ranges from about 1:0.2 to about 1:0.5, and in at least one embodiment is about 1:0.25. In a still further embodiment, a composition according to the disclosure comprises from about 4% to about 6% by weight of β-cyclodextrin, and a solvent comprising water and at least one additional solvent chosen from hexylene glycol, pentylene glycol, or a mixture thereof, where weight ratio of cyclodextrin:additional solvent ranges from about 1:0.2 to about 1:0.5, and in at least one embodiment is about 1:0.25.

In other embodiments, compositions according to the disclosure comprise: (a) at least one cyclodextrin; (b) at least one solvent; (c) at least one cationic surfactant; (c) at least one emulsifier; (d) at least one silicone; and (e) at least one oil. In some embodiments, the at least one solvent comprises water and at least one additional solvent that can improve the solubility of the at least one cyclodextrin, and the weight ratio of cyclodextrin:additional solvent ranges from about 1.2:0.1 to about 1:1, such as from about 1:0.1 to about 1:0.8, from about 1:0.15 to about 1:0.6, or about 1:0.2 to about 1:0.5. For example, the weight ratio of cyclodextrin:additional solvent may be about 1:0.25. In various embodiments, the at least one additional solvent is chosen from linear or branched, saturated or unsaturated, and substituted or unsubstituted C₃-C₈ polyols, such as, for example, hexylene glycol, pentylene glycol, or a mixture thereof. However, in at least some embodiments, no additional solvent is needed to improve the solubility of the at least one cyclodextrin or derivative thereof, although it may be desirable to include one or more of these solvents as part of the solvent mixture.

In various embodiments, the at least one cyclodextrin may be chosen from those of formula:

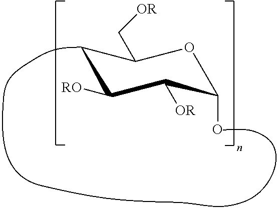

where R is chosen from H, CH₃, or a hydroxypropyl group, and n ranges from 6-8. For example, the at least one cyclodextrin or derivative thereof may be α-cyclodextrin, β-cyclodextrin, γ-cyclodextrin, a methyl derivative of α-cyclodextrin, β-cyclodextrin, or γ-cyclodextrin, a hydroxypropyl derivative of α-cyclodextrin, β-cyclodextrin, or γ-cyclodextrin, or a mixture of any two or more of the foregoing. The at least one cyclodextrin or derivative thereof may be present in an amount ranging from about 0.01% to about 20%, such as from about 0.1% to about 18%, from about 0.5% to about 15%, or from about 1% to about 13% by weight, relative to the total weight of the composition, for example from about 0.5% to about 10%, from about 1% to about 8%, from about 3% to about 7%, or from about 4% to about 6%.

In various embodiments, the at least one oil is a low molecular weight oil. For example, the at least one oil may be chosen from saturated or unsaturated plant oils having less than 16 carbon atoms, for example coconut oil, shea butter, castor oil, palmitic acid, jojoba oil, or a mixture of any two or more of the foregoing. In certain embodiments, the weight ratio of cyclodextrin:oil ranges from about 1.2:0.5 to about 1:2, such as about 1:0.75 to about 1:1.75, about 1:1.25 to about 1:1.5, or about 1:0.75 to about 1:1.5.

In some embodiments, a composition according to the disclosure comprises from about 0.5% to about 10%, from about 1% to about 8%, from about 3% to about 7%, or from about 4% to about 6% by weight of at least one cyclodextrin chosen from α-cyclodextrin, β-cyclodextrin, γ-cyclodextrin, or a mixture of two or more of the foregoing, at least one solvent, at least one cationic surfactant, at least one emulsifier, at least one silicone, and at least one oil chosen from saturated or unsaturated plant oils having less than 16 carbon atoms, where the weight ratio of cyclodextrin:oil ranges from about 1:0.75 to about 1:1.75, about 1:1.25 to about 1:1.5, or about 1:0.75 to about 1:1.5.

In an embodiment, a composition according to the disclosure comprises at least one cyclodextrin chosen from α-cyclodextrin, β-cyclodextrin, γ-cyclodextrin, or a mixture of two or more of the foregoing, at least one solvent, at least one cationic surfactant, at least one emulsifier, at least one silicone, and at least one oil chosen from saturated or unsaturated plant oils having less than 16 carbon atoms, for example coconut oil, shea butter, castor oil, palmitic acid, jojoba oil, or a mixture of any two or more of the foregoing, where the weight ratio of cyclodextrin:oil ranges from about 1:0.75 to about 1:1.75, about 1:1.25 to about 1:1.5, or about 1:0.75 to about 1:1.5. In another embodiment, a composition according to the disclosure comprises from about 3% to about 7% by weight of at least one cyclodextrin chosen from α-cyclodextrin, β-cyclodextrin, γ-cyclodextrin, or a mixture of two or more of the foregoing, at least one solvent, at least one cationic surfactant, at least one emulsifier, at least one silicone, and from about 0.1% to about 10% by weight, such as from about 2% to about 8% of at least one oil chosen from saturated or unsaturated plant oils having less than 16 carbon atoms, for example coconut oil, shea butter, castor oil, palmitic acid, jojoba oil, or a mixture of any two or more of the foregoing, where the weight ratio of cyclodextrin:oil ranges from about 1:0.75 to about 1:1.75, about 1:1.25 to about 1:1.5, or about 1:0.75 to about 1:1.5. In a still further embodiment, a composition according to the disclosure comprises from about 4% to about 6% by weight of β-cyclodextrin or a derivative thereof, water, at least one cationic surfactant, at least one emulsifier, at least one silicone, and from about 2% to about 8% of at least one oil chosen from coconut oil, shea butter, castor oil, palmitic acid, jojoba oil, or a mixture of any two or more of the foregoing, where the weight ratio of cyclodextrin:oil ranges from about 1:1.25 to about 1:1.5.

In yet a further embodiment, a composition according to the disclosure comprises at least one cyclodextrin chosen from α-cyclodextrin, β-cyclodextrin, γ-cyclodextrin, a methyl or hydroxypropyl derivative of α-cyclodextrin, β-cyclodextrin, or γ-cyclodextrin, or a mixture of two or more of the foregoing, at least one solvent, and at least one additional component chosen from cationic surfactants, emulsifiers, silicones, and/or oils chosen from saturated or unsaturated plant oil having less than 16 carbon atoms. In a further embodiment, a composition according to the disclosure comprises from about 0.5% to about 10%, from about 1% to about 8%, from about 3% to about 7%, or from about 4% to about 6% by weight of at least one cyclodextrin chosen from α-cyclodextrin, β-cyclodextrin, γ-cyclodextrin, a methyl or hydroxypropyl derivative of α-cyclodextrin, β-cyclodextrin, or γ-cyclodextrin, or a mixture of two or more of the foregoing, at least one solvent, and at least one additional component chosen from cationic surfactants, emulsifiers, silicones, and/or oils chosen from saturated or unsaturated plant oil having less than 16 carbon atoms. In a still further embodiment, a composition according to the disclosure comprises from about 0.5% to about 10%, from about 1% to about 8%, from about 3% to about 7%, or from about 4% to about 6% by weight of at least one cyclodextrin chosen from α-cyclodextrin, β-cyclodextrin, γ-cyclodextrin, a methyl or hydroxypropyl derivative of α-cyclodextrin, β-cyclodextrin, or γ-cyclodextrin, or a mixture of two or more of the foregoing, at least one solvent, from about 0.1% to about 10% by weight, such as from about 2% to about 8% by weight of at least one oil chosen from saturated or unsaturated plant oils having less than 16 carbon atoms, for example coconut oil, shea butter, castor oil, palmitic acid, jojoba oil, or a mixture of any two or more of the foregoing, where the weight ratio of cyclodextrin:oil ranges from about 1:0.75 to about 1:1.75, about 1:1.25 to about 1:1.5, or about 1:0.75 to about 1:1.5, and optionally at least one additional component chosen from cationic surfactants, emulsifiers, and/or silicones.

In further embodiments, a composition according to the disclosure comprises from about 0.5% to about 10%, from about 1% to about 8%, from about 3% to about 7%, or from about 4% to about 6% by weight of at least one cyclodextrin chosen from α-cyclodextrin, β-cyclodextrin, γ-cyclodextrin, a methyl or hydroxypropyl derivative of α-cyclodextrin, β-cyclodextrin, or γ-cyclodextrin, or a mixture of two or more of the foregoing, at least one solvent comprising water and at least one additional solvent that can improve the solubility of the at least one cyclodextrin, wherein the weight ratio of cyclodextrin:additional solvent ranges from about 1.2:0.1 to about 1:1, such as from about 1:0.1 to about 1:0.8, from about 1:0.15 to about 1:0.6, or about 1:0.2 to about 1:0.5, at least one cationic surfactant, at least one emulsifier, at least one silicone, and at least one oil chosen from saturated or unsaturated plant oils having less than 16 carbon atoms, where the weight ratio of cyclodextrin:oil ranges from about 1:0.75 to about 1:1.75, about 1:1.25 to about 1:1.5, or about 1:0.75 to about 1:1.5. In a still further embodiment, a composition according to the disclosure comprises from about 0.5% to about 10%, from about 1% to about 8%, from about 3% to about 7%, or from about 4% to about 6% by weight of at least one cyclodextrin chosen from α-cyclodextrin, β-cyclodextrin, γ-cyclodextrin, a methyl or hydroxypropyl derivative of α-cyclodextrin, β-cyclodextrin, or γ-cyclodextrin, or a mixture of two or more of the foregoing, at least one solvent comprising water and at least one additional solvent that can improve the solubility of the at least one cyclodextrin, wherein the weight ratio of cyclodextrin:additional solvent ranges from about 1.2:0.1 to about 1:1, such as from about 1:0.1 to about 1:0.8, from about 1:0.15 to about 1:0.6, or about 1:0.2 to about 1:0.5, and at least one additional component chosen from cationic surfactants, emulsifiers, silicones, and/or oils chosen from saturated or unsaturated plant oil having less than 16 carbon atoms. In a further embodiment, a composition according to the disclosure comprises from about 0.5% to about 10%, from about 1% to about 8%, from about 3% to about 7%, or from about 4% to about 6% by weight of at least one cyclodextrin chosen from α-cyclodextrin, β-cyclodextrin, γ-cyclodextrin, a methyl or hydroxypropyl derivative of α-cyclodextrin, β-cyclodextrin, or γ-cyclodextrin, or a mixture of two or more of the foregoing, at least one solvent comprising water and at least one additional solvent that can improve the solubility of the at least one cyclodextrin, wherein the weight ratio of cyclodextrin:additional solvent ranges from about 1.2:0.1 to about 1:1, such as from about 1:0.1 to about 1:0.8, from about 1:0.15 to about 1:0.6, or about 1:0.2 to about 1:0.5, from about 0.1% to about 10% by weight, such as from about 2% to about 8% by weight of at least one oil chosen from saturated or unsaturated plant oils having less than 16 carbon atoms, for example coconut oil, shea butter, castor oil, palmitic acid, jojoba oil, or a mixture of any two or more of the foregoing, where the weight ratio of cyclodextrin:oil ranges from about 1:0.75 to about 1:1.75, about 1:1.25 to about 1:1.5, or about 1:0.75 to about 1:1.5, and optionally at least one additional component chosen from cationic surfactants, emulsifiers, and/or silicones.

In an embodiment, a composition according to the disclosure comprises from about 3% to about 7% by weight of at least one cyclodextrin chosen from α-cyclodextrin, β-cyclodextrin, γ-cyclodextrin, or a mixture of two or more of the foregoing, at least one solvent comprising water and at least one additional solvent that can improve the solubility of the at least one cyclodextrin, wherein the weight ratio of cyclodextrin:additional solvent ranges from about 1:0.15 to about 1:0.6 or about 1:0.2 to about 1:0.5, from about 0.1% to about 10% by weight of at least one oil where the weight ratio of cyclodextrin:oil ranges from about 1:1.25 to about 1:1.5, and optionally at least one additional component chosen from cationic surfactants, emulsifiers, and/or silicones. In another embodiment, a composition according to the disclosure comprises from about 3% to about 7% by weight of at least one cyclodextrin chosen from α-cyclodextrin, β-cyclodextrin, γ-cyclodextrin, or a mixture of two or more of the foregoing, at least one solvent comprising water and at least one additional solvent that can improve the solubility of the at least one cyclodextrin, wherein the weight ratio of cyclodextrin:additional solvent ranges from about 1.2:0.1 to about 1:1, such as from about 1:0.2 to about 1:0.5, from about 0.1% to about 10% by weight of at least one oil chosen from saturated or unsaturated plant oils having less than 16 carbon atoms, for example coconut oil, shea butter, castor oil, palmitic acid, jojoba oil, or a mixture of any two or more of the foregoing, where the weight ratio of cyclodextrin:oil ranges from about 1:0.75 to about 1:1.75, about 1:1.25 to about 1:1.5, or about 1:0.75 to about 1:1.5, and optionally at least one additional component chosen from cationic surfactants, emulsifiers, and/or silicones. In one embodiment, a composition according to the disclosure comprises from about 1% to about 10% by weight of β-cyclodextrin or a derivative thereof, at least one solvent comprising water and at least one additional solvent that can improve the solubility of the at least one cyclodextrin, wherein the weight ratio of cyclodextrin:additional solvent ranges from about 1.2:0.1 to about 1:1, such as from about 1:0.2 to about 1:0.5, from about 0.1% to about 10% by weight of at least one oil chosen from coconut oil, shea butter, castor oil, palmitic acid, jojoba oil, or a mixture of any two or more of the foregoing, where the weight ratio of cyclodextrin:oil ranges from 1:1.25 to about 1:1.5, or about 1:0.75 to about 1:1.5, and optionally at least one additional component chosen from cationic surfactants, emulsifiers, and/or silicones. In a still further embodiment, a composition according to the disclosure comprises from about 1% to about 10% by weight of β-cyclodextrin or a derivative thereof, at least one solvent comprising water and at least one additional solvent that can improve the solubility of the at least one cyclodextrin, wherein the weight ratio of cyclodextrin:additional solvent ranges from about 1.2:0.1 to about 1:1, such as from about 1:0.2 to about 1:0.5, from about 0.1% to about 10% by weight of at least one oil chosen from coconut oil, shea butter, castor oil, palmitic acid, jojoba oil, or a mixture of any two or more of the foregoing, where the weight ratio of cyclodextrin:oil ranges from 1:1.25 to about 1:1.5, or about 1:0.75 to about 1:1.5, at least one cationic surfactant, at least one emulsifier, and at least one silicone.

The disclosure further relates to methods of using the compositions, for example methods of treating hair, methods of elongating curls, methods of lengthening the visual appearance of hair, and/or methods of reducing frizz and/or volume of the hair, wherein the methods comprise applying the composition to wet, damp, or dry hair. Optionally, the compositions are left on the hair for a period of a few hours to a few days, for example until the hair is washed. The methods may provide one or more benefits to the hair, such as curl elongation, frizz control, smoothness, and/or softness.

### BRIEF DESCRIPTION OF THE FIGURES

The accompanying drawings, which are incorporated herein and constitute part of this specification, illustrate exemplary embodiments of the disclosure, and, together with the general description given above and the description provided herein, serve to explain features of the disclosure.

FIG. 1 is a graph demonstrating the difference in elongation over a period of 24 hours of compositions C1-C2 and 1A-1E.

FIG. 2 shows images of hair swatches treated with compositions C1-C2 and 1A-1E at T24H.

FIG. 3 shows images of half-head comparisons of compositions 2D and C3-C4.

FIG. 4 is a graph which demonstrates the curl shrinkage of compositions 3A-3F compared to that of composition C5.

FIG. 5 is a graph which demonstrates the curl shrinkage of compositions 4A-4F compared to that of composition C6.

It is to be understood that the foregoing and following descriptions are exemplary and explanatory only, and are not intended to be restrictive of any subject matter claimed.

### DETAILED DESCRIPTION

The disclosure relates to compositions for styling hair, such as for elongation of curls and/or reducing the degree of curliness of hair, as well as to methods of using the compositions.

### I. Compositions

In various embodiments, compositions according to the disclosure comprise (a) at least one cyclodextrin or derivative thereof; (b) at least one solvent; (c) optionally at least one cationic surfactant; (c) optionally at least one emulsifier; (d) optionally at least one silicone; and (e) optionally at least one oil.

Compositions according to the disclosure may improve and/or maintain elongation of curly or wavy hair, and in at least some embodiments may provide additional benefits such as softness, smoothness, and/or frizz control to the hair.

### Cyclodextrins

Compositions according to the disclosure comprise at least one cyclodextrin, which includes derivatives thereof. Cyclodextrins are a family of cyclic oligosaccharides consisting of a macrocyclic ring of glucose subunits joined by α-1,4 glycosidic bonds.

The cyclodextrins that can be used include those of the formula:

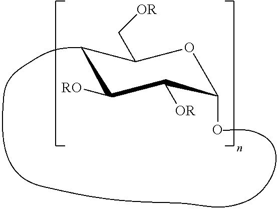

wherein:
R is chosen from H, CH₃, or a hydroxypropyl group, and
n ranges from 6-8.

For example, in embodiments where R=H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R=CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodextrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

The at least one cyclodextrin may be present in the compositions in an amount of about 0.01% to about 20% by weight, such as about 0.1% to about 18%, about 0.5% to about 15%, or about 1% to about 13% by weight, relative to the total weight of the composition. By way of non-limiting example, the at least one cyclodextrin is present in the composition with a total amount ranging from about 0.5% to about 15%, by weight, for example, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 15%, from about 2% to about 12%, from about 2% to about 10%, from about 2% to about 9%, from about 2% to about 8%, from about 2% to about 7%, from about 2% to about 6%, or from about 2% to about 5% by weight, relative to the total weight of the composition, including all ranges and subranges thereof.

In one embodiment, the at least one cyclodextrin comprises or consists of β-cyclodextrin, and is present in an amount ranging from about 0.5% to about 10%, such as about 1% to about 8%, about 3% to about 7%, or about 4% to about 6% by weight, relative to the total weight of the composition, including all ranges and subranges thereof.

### Solvents

Compositions according to the disclosure comprise one or more cosmetically acceptable solvents. The solvents may be chosen from water, non-aqueous solvents, or mixtures thereof.

In one embodiment, the solvent comprises water. In other embodiments, the composition comprises one or more non-aqueous solvents. For example, C₁₋₄ alcohols, organic solvents, fatty alcohols, fatty ethers, fatty esters, polyols, glycols, vegetable oils, mineral oils, liposomes, laminar lipid materials, or any mixture thereof. Non-limiting examples of solvents which may be used include alkane polyols, such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-isopropyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-isopropyl ether, dipropylene glycol monomethyl ether, dipropylene glycol monoethyl ether, dipropylene glycol mono-n-propyl ether, and dipropylene glycol mono-isopropyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In some exemplary embodiments, the solvent comprises water and at least one additional solvent that can improve the solubility of the at least one cyclodextrin. For example, the at least one additional solvent may be chosen from linear or branched, saturated or unsaturated, and substituted or unsubstituted polyols, for example C₃-C₈ polyols or C₄-C₆ polyols, such as diols and/or triols. Any stereoisomer of the polyols may be used.

In various exemplary embodiments, the C₃-C₈ polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, and mixtures thereof.

In a preferred embodiment, the at least one additional solvent comprises, consists essentially of, or consists of hexylene glycol, pentylene glycol, or a mixture thereof.

In various embodiments, the at least one additional solvent that can improve the solubility of the at least one cyclodextrin can be present in the composition in a total amount ranging from about 0.01% to about 10%, such as from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1.5%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1.5%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, or from about 1% to about 1.5% by weight, relative to the total weight of the composition, including all ranges and subranges thereof.

In some embodiments, the amount of the at least one additional solvent that can improve the solubility of the at least one cyclodextrin may be chosen such that it is present in a particular weight ratio of cyclodextrin:additional solvent, for example ranging from about 1.2:0.1 to about 1:1, such as from about 1:0.1 to about 1:0.8, from about 1:0.15 to about 1:0.6, or about 1:0.2 to about 1:0.5, including all ranges and subranges thereof. For example, the ratio of cyclodextrin:additional solvent may be about 1:0.1, about 1:0.15, about 1:0.2, about 1:0.25, about 1:0.3, about 1:0.35, about 1:0.4, or about 1:0.5, including all ranges and subranges thereof.

The total amount of solvent in the composition may range from about 50% to about 99% by weight, relative to the total weight of the composition, including all ranges and subranges therebetween. For example, in one embodiment, the total amount of solvent may be about 50% to about 98%, about 60% to about 98%, about 70% to about 98%, about 70% to about 95%, about 75% to 95%, or about 80% to 95% by weight, relative to the total weight of the composition. In certain embodiments, the solvent is primarily comprised of water, such as from about 90% to about 99%, or about 95% to about 99.9%, of the total solvent, with the remainder of the solvent comprising one or more additional solvents that can improve the solubility of the at least one cyclodextrin. In other embodiments, the solvent comprises, consists essentially of, or consists of water, and no additional solvent is needed to improve the solubility of the at least one cyclodextrin. However, it should be understood that such additional solvents can be included in the solvent mixture whether or not the solubility of the at least one cyclodextrin is improved.

### Cationic Surfactants

Compositions according to the disclosure optionally comprise at least one cationic surfactant. The term “cationic surfactant” means a surfactant comprising, as ionic or ionizable groups, only cationic groups. In certain embodiments, the cationic surfactants are plant-based and/or organic.

In some embodiments, a cationic surfactant may be chosen from amidoamine compounds (or amidoamines). Examples of amidoamines that are useful in the compositions of the instant disclosure include, but are not limited to the following: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidoethyldiethylamine, and mixtures thereof.

In some further embodiments, a cationic surfactant can be chosen from monoalkyl quaternary amines, dialkyl quaternary amines, or polyquaternium compounds or salts thereof.

For example, a cationic surfactant may be chosen from Polyquaterium-10 (also called quaternized polyhydroxyethyl cellulose), cetrimonium chloride, behentrimonium chloride, behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Polyquaternium-37 (e.g., under the SALCARE tradename), Quaternium-22, Quaternium-87, Polyquaternium-4, Polyquaternium-6, Polyquaternium-11, Polyquaternium-44, Polyquaternium-67, amodimethicone, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, aminopropyl dimethicone, Quaterium-8, and dilinoleamidopropyl dimethylamine dimethicone PEG-7 phosphate.

In some embodiments, a suitable cationic surfactant may be chosen from polyquaternium 4, polyquaternium 6, polyquaternium 7, polyquaternium 10, polyquaternium 11, polyquaternium 16, polyquaternium 22, polyquaternium 28, polyquaternium-32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, or guar hydroxypropyltrimonium chloride, and mixtures thereof. In an embodiment, the cationic surfactant is chosen from Polyquaternium-67, Polyquaternium-10, Polyquaternium-37, or mixtures thereof. Polyquaternium-37 may be commercially available from BASF under the tradename of SALCARE SC 96 (comprising Polyquaternium-37 (and) Propylene Glycol Dicaprylate/Dicaprate (and) PPG-1 Trideceth-6).

In some embodiments, the at least one cationic surfactant in the compositions comprises behentrimonium chloride. In some further embodiments, the at least one cationic surfactant is chosen from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyl-diethylamine, behenamidoethyldimethylamine, arachidamidopropyldimethylamine, arachidamido-propyidiethylamine, arachidamidoethyldiethylamine, arachidamidoethyl-dimethylamine, or a combination thereof.

Non-limiting examples of cationic surfactants may also include behenalkonium chloride, benzethonium chloride, cetylpyridinium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

In some embodiments, the at least one cationic surfactant is chosen from polyoxyalkylenated primary, secondary, or tertiary fatty amine salts, quaternary ammonium salts, or mixtures thereof. In some cases it is useful to use salts such as hloride salts of the quaternary ammonium compounds. The fatty amines may comprise at least one Cs-Ca) hydrocarbon-based chain.

In some embodiments, suitable quaternary ammonium salts may be those of formula (I):

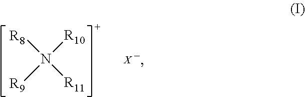

wherein:
groups R8 to R11 are independently chosen from linear or branched aliphatic groups containing from 1 to 30 carbon atoms, or an aromatic group such as aryl or alkylaryl, at least one of the groups R8 to R11 including from 8 to 30 carbon atoms, such as from 12 to 24 carbon atoms, it being possible for the linear or branched aliphatic groups to include heteroatoms such as, for example, oxygen, nitrogen, and/or sulfur, these heteroatoms not being adjacent, and halogens; and
X⁻ is an anion chosen from the group consisting of halides such as bromides, chlorides, iodides, fluorides, phosphates, acetates, lactates, (C1-C4)alkyl sulfates, (C1-C4)alkyl sulfonates or (C1-C4)alkylaryl sulfonates; C1-C30 alkyl, C1-C30 alkoxy, (C2-C6)polyoxyalkylene, C1-C30 alkylamide, (C12-C22)alkyl-(C2C6)alkylamido, (C12-C22)alkyl acetate, and C1-C30 hydroxyalkyl groups.

Mention may be made as exemplary embodiments of formula (V) of tetraalkylammonium halides, such as chlorides, for example dialkyldimethylammonium or alkyltrimethylammonium chlorides in which the alkyl group comprises from 12 to 22 carbon atoms, such as from 14 to 20 carbon atoms. By way of example, behenyltrimethylammonium chloride (behentrimonium chloride), distearyl-dimethylammonium chloride, cetyltrimethylammonium chloride (cetrimonium chloride), or benzyldimethylstearylammonium chloride may be chosen.

In some embodiments, compounds of formula (I) may include palmitylamidopropyltrimethylammonium or stearamidopropyldimethyl-(myristyl acetate)-ammonium halides, such as chlorides, for example the product sold under the name CERAPHYL® 70 by the company Van Dyk.

In certain embodiments, cationic surfactants of formula (I) are preferably chosen from alkyltrimethylammonium halides whose alkyl group includes from 12 to 22 carbon atoms, such as from 14 to 20 carbon atoms, may be chosen. For example, alkyltrimethylammonium chlorides, such as behenyltrimethylammonium chloride and cetyltrimethylammonium chloride, may be particularly useful.

In further embodiments, quaternary ammonium salts of imidazoline of formula (II) may be chosen:

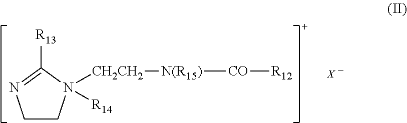

wherein:
R12 represents an alkenyl or alkyl group comprising from 8 to 30 carbon atoms, for example derived from tallow fatty acids,
R13 represents a hydrogen atom, a C1-C4 alkyl group or an alkenyl or alkyl group comprising from 8 to 30 carbon atoms,
R14 represents a C1-C4 alkyl group,
R15 represents a hydrogen atom or a C1-C4 alkyl group, and
X⁻ is an anion chosen from the group consisting of halides, phosphates, acetates, lactates, (C1-C4)alkyl sulfates, and (C1-C4)alkyl- or (C1-C4)alkylarylsulfonates.

In one exemplary embodiment of formula (II), R12 and R13 represent a mixture of alkenyl or alkyl groups containing from 12 to 21 carbon atoms, derived for example from tallow fatty acids; R14 represents a methyl group; and R15 represents a hydrogen atom. Such a product may be sold, for example, under the name REWOQUAT® W 75 by the company Evonik.

In yet further embodiments, di- or triquaternary ammonium salts of formula (III) may be chosen:

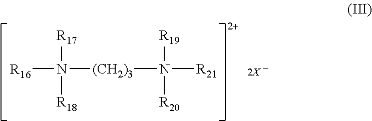

wherein:
R16 represents an alkyl group comprising from 16 to 30 carbon atoms, which is optionally hydroxylated and/or optionally interrupted with one or more oxygen atoms,
R17 represents hydrogen, an alkyl group comprising from 1 to 4 carbon atoms, or a group —(CH2)3-N+(R16a)(R17a)(R18a),
R16a, R17a and R18a, which may be identical or different, represent hydrogen or an alkyl group comprising from 1 to 4 carbon atoms,
R18, R19, R20, and R21, which may be identical or different, represent hydrogen or an alkyl group comprising from 1 to 4 carbon atoms, and
X⁻ is an anion chosen from the group consisting of halides, acetates, phosphates, nitrates, (C1-C4)alkyl sulfates, and (C1-C4)alkyl- and (C1-C4)alkylarylsulfonates, for example methyl sulfate or ethyl sulfate.

Such compounds are, for example, FINQUAT® CT-P (Quaternium 89) and FINQUAT® CT (Quaternium 75), sold by the company Finetex.

In still further embodiments, quaternary ammonium salts containing one or more ester functions, such as those of formula (IV) may be chosen:

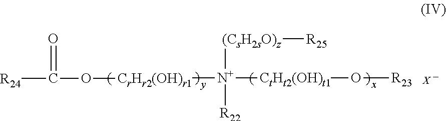

wherein:

R22 is chosen from C1-C6 alkyl groups and C1-C6 hydroxyalkyl or dihydroxyalkyl groups,
R23 is chosen from the group R26-C(═O)—; linear or branched, saturated or unsaturated C1-C22 hydrocarbon-based groups; and a hydrogen atom,
R25 is chosen from the group R28-C(═O)—; linear or branched, saturated or unsaturated C1-C6 hydrocarbon-based groups; and a hydrogen atom,
R24, R26, and R28, which may be identical or different, are chosen from saturated or unsaturated, linear or branched C7-C21 hydrocarbon-based groups,
r, s, and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10, and
X⁻ is an anion;
it being understood that r2+r1=2r and t1+t2=2t, and that the sum x+y+z ranges from 1 to 15, with the proviso that when x=0 then R23 is chosen from C1-C22 hydrocarbon-based groups, and that when z=0 then R25 denotes a linear or branched, saturated or unsaturated C1-C6 hydrocarbon-based group.

In exemplary embodiments of formula (IV), the alkyl groups R22 may be linear or branched, and are preferably linear. Preferably, R22 denotes a methyl, ethyl, hydroxyethyl or dihydroxypropyl group, and more particularly a methyl or ethyl group. Advantageously, the sum x+y+z ranges from 1 to 10. When R23 is a C1-C22 hydrocarbon-based groups, it may preferably comprise either from 12 to 22 carbon atoms or from 1 to 3 carbon atoms. Advantageously, R24, R26 and R28, which may be identical or different, are chosen from linear or branched, saturated or unsaturated C11-C21 hydrocarbon-based groups, and more particularly from linear or branched C11-C21 alkyl and alkenyl groups. Preferably, x and z, which may be identical or different, are equal to 0 or 1. Optionally, y is equal to 1. Preferably, r, s, and t, which may be identical or different, are equal to 2 or 3, and optionally are equal to 2.

The anion X⁻ is preferably a halide, optionally chloride, bromide, or iodide, a (C1-C4)alkyl sulfate, a (C1-C4)alkylsulfonate, or a (C1-C4)alkylarylsulfonate, a methanesulfonate, a phosphate, a nitrate, a tosylate, an anion derived from an organic acid such as an acetate or a lactate, or any other anion that is compatible with the ammonium bearing an ester function. In some embodiments, the anion X− is a chloride, a methyl sulfate, or an ethyl sulfate.

For example, the ammonium salts of formula (IV), in which R22 is a methyl or ethyl group, x and y are equal to 1, z is equal to 0 or 1, r, s and t are equal to 2, R23 is chosen from the group R26-C(═O)—, methyl, ethyl, or C14-C22 hydrocarbon-based groups, and a hydrogen atom; R25 is chosen from the group R28 -C(═O)—, and a hydrogen atom; R24, R26 and R28, which may be identical or different, are chosen from linear or branched, saturated or unsaturated C13-C17 hydrocarbon-based groups, and preferably from linear or branched, saturated or unsaturated C13-C17 alkyl and alkenyl groups, may be chosen. In one embodiment, the hydrocarbon-based groups are linear.

Among the compounds having formula (IV), mention may be made of salts, especially the chloride or methyl sulfate salts, of diacyloxyethyldimethylammonium, diacyloxyethylhydroxyethylmethylammonium, monoacyloxyethyldihydroxyethylmethyl-ammonium, triacyloxyethylmethylammonium or monoacyloxyethylhydroxyethyldimethyl-ammonium, and mixtures thereof. The acyl groups preferably contain 14 to 18 carbon atoms and are obtained more particularly from a plant oil such as palm oil or sunflower oil. When the compound contains several acyl groups, these groups may be identical or different.

These cationic surfactants may be obtained, for example, by direct esterification of triethanolamine, triisopropanolamine, alkyldiethanolamine or alkyldiisopropanolamine, which are optionally oxyalkylenated, with fatty acids or with fatty acid mixtures, such as those of plant or animal origin, or by transesterification of the methyl esters thereof. This esterification may be followed by a quaternization by means of an alkylating agent, such as an alkyl halide, for example, methyl or ethyl halide, a dialkyl sulfate, for example, dimethyl or diethyl sulfate, methyl methanesulfonate, methyl para-toluenesulfonate, glycol chlorohydrin, or glycerol chlorohydrin. Such compounds are sold, for example, under the names DEHYQUART® by the company Henkel, STEPANQUAT® by the company Stepan, NOXAMIUM® by the company CECA or REWOQUAT® WE 18 by the company Evonik.

In some embodiments, the at least one cationic surfactant may be chosen from, for example, a mixture of quaternary ammonium monoester, diester and triester salts with a weight majority of diester salts. In some embodiment, the at least one cationic surfactant may be chosen from the ammonium salts containing at least one ester functional group that are described in patents U.S. Pat. Nos. 4,874,554 and 4,137,180, which are incorporated herein for their entireties. In some embodiment, the at least one cationic surfactant may comprise behenoylhydroxypropyl-trimethylammonium chloride, for example, sold by the company Kao under the name QUARTAMIN® BTC 131.

Suitable cationic surfactants may further include those having a structure in accordance with the general formula provided below:

R4-A-R5-B (V),

wherein R4 is a saturated or unsaturated, straight or branched alkyl chain with 8 to 24 carbon atoms, R5 is a straight or branched alkyl chain with 1 to 4 carbon atoms, A is selected from:

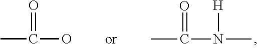

and B is selected from compounds of formula (VI):

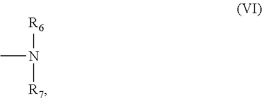

wherein R₆ and R₇, which may be the same or different, are chosen from H, an alkyl chain with 1 to 4 carbon atoms, hydroxyl alkyl chain with 1 to 4 carbon atoms, or di hydroxyl alkyl chain with 2 to 4 carbon atoms; or compounds of formula (VII):

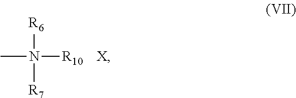

wherein R₈ and R₉, which can be the same or different, are chosen from an alkyl chain with 1 to 4 carbon atoms, a hydroxyl alkyl chain with 1 to 4 carbon atoms, or a di hydroxyl alkyl chain with 2 to 4 carbon atoms; wherein R₁₀ is chosen from an alkyl chain with 1 to 4 carbon atoms, a hydroxyl alkyl chain with 1 to 4 carbon atoms, or a di hydroxyl alkyl chain with 2 to 4 carbon atoms.

In some instances, in formula (V), R₄ is saturated or unsaturated, straight or branched alkyl chain with 10 to 24 carbon atoms, more preferably 12 to 22 carbon atoms and R₅ is straight or branched alkyl group with 1 to 4 carbon atoms, and A, B, R₆ to R₁₀ are same as above.

Non-limiting suitable examples include stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl buylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl prpylamine, dipalmitamidopropyl hydroxyethylamine, dipalmitamidopropyl hydroxypropylamine, dilauramidopropyl amine, dilauramidopropyl methylamine, dilauramidopropyl butylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimthyl ammonium chloride, dicetylamidodihydroxyethyl ammonium chloride, palmitoylpropyl amine, palmitoylpropyl methylamine, palmitoylpropyl diethylamine, palmitoylpropyl dibutylamine, palmitoylpropyl buylamine, palmitoylpropyl dipropylamine, palmitoylpropyl propylamine, palmidopropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl butylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitoylpropyl butylamine, dipalmitoylpropyl propylamine, dipalmitoylpropyl hydrosyethylamine, dipalmitoylpropyl hydroxypropylamine, dilauroylpropyl amine, dipalmitoylpropyl methylamine, dipalmitoylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

In some embodiments, a cationic surfactant may be chosen from cationizable surfactants such as fatty alkylamines, preferably, fatty dialkylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and mixtures thereof.

Fatty dialkylamines include fatty amidoamine compounds, their salts, and mixtures thereof. Non-limiting examples include oleamidopropyl dimethylamine, linoleamidopropyl dimethylamine, isostearamidopropyl dimethylamine, stearamidopropyl dimethylamine, oleyl hydroxyethyl imidazoline, stearamidopropyldimethylamine, behenamidopropyldimethylamine, behenamidopropyldiethylamine, behenamidoethyl-diethylamine, behenamidoethyldimethylamine, arachidamidopropyldimethylamine, arachidamido-propyidiethylamine, arachidamidoethyldiethylamine, arachidamidoethyl-dimethylamine, brassicamidopropyldimethylamine, lauramidopropyl dimethylamine, myristamidopropyl dimethylamine, dilinoleamidopropyl dimethylamine, and palmitamidopropyl dimethylamine.

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. In some embodiment, a carboxylic acid may be chosen from lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, or mixtures thereof. In some embodiment, lactic acid, tartaric acid or mixtures thereof are used, especially in combination with fatty dimethylamines such as, for example, stearamidopropyl dimethylamine.

Compositions according to the disclosure may be formulated such that two or more cationic surfactants are associated with the same or different balancing anionic ions. For example, at least one of the two or more cationic surfactants may have a chloride ion and/or a sulfate ion. In some embodiments, the two or more cationic surfactants are chosen from cetrimonium chloride, behentrimonium methosulfate, behentrimonium chloride, or combination thereof. In some further embodiments, the two or more cationic surfactants comprise behentrimonium chloride and one or both of behentrimonium methosulfate and cetrimonium chloride.

If present, in various embodiments, the total amount of the at least one cationic surfactant may range up to about 15%, based on the total weight of the composition, including all ranges and subranges therebetween. For instance, the total amount of the at least one cationic surfactant may range from about 0.01% to about 10%, from about 0.1% to about 8%, from about 0.5% to about 5%, or from about 0.75% to about 3% by weight, relative to the total weight of the composition.

### Emulsifiers

Compositions according to the disclosure optionally comprise at least one emulsifier. In various embodiments, the one or more emulsifiers may be chosen from fatty alcohols, esters, or a combination thereof.

### Fatty Alcohols

In some embodiments, compositions according to the disclosure comprise at least one emulsifier chosen from fatty alcohols, or derivatives thereof. In certain embodiments, the compositions comprise at least two fatty alcohols.

As used herein, “fatty alcohol” refers to any alcohol with a carbon chain of C5 or greater, such as, for example, C8 or greater, C10 or greater, and C12 or greater. Suitable fatty alcohols according to the disclosure may include, but are not limited to, alkoxylated or non-alkoxylated, saturated or unsaturated, linear or branched, fatty alcohols, for example with from 6 to 30 carbon atoms, such as from 8 to 30 carbon atoms, from 8 to 22 carbon atoms, from 12 to 22 carbon atoms, or from 12 to 18 carbon atoms, including all ranges and subranges therebetween. The fatty alcohols may be liquid or solid. Non-limiting examples of the fatty alcohols and derivatives thereof are found in International Cosmetic Ingredient Dictionary, Sixteenth Edition, 2016, which is incorporated by reference herein in its entirety.

As used herein, “alkoxylated fatty alcohol” refers to any fatty alcohol with a carbon chain of C5 or greater, as defined above, further comprising at least one alkoxy group. For example, the at least one alkoxylated fatty alcohol may have a carbon chain of C8 or greater, C10 or greater, and C12 or greater. Further, for example, the at least one alkoxylated fatty alcohol may be chosen from alkoxylated polymers (including co-, ter- and homo-polymers) derived from alcohols such as glycerol (e.g., polyglyceryl derived from four glycerol molecules). The at least one alkoxy group of the at least one alkoxylated fatty alcohol may, for example, be derived from an alkoxylation reaction carried out with alkylene oxide. Non-limiting examples of at least one alkoxylated fatty alcohol include any fatty alcohol comprising at least one polyethylene glycol ether and any fatty alcohol comprising at least one polypropylene glycol ether.

In some embodiments, the compositions according to the disclosure comprise a liquid fatty alcohol. In some embodiments, the compositions according to the disclosure comprise a solid fatty alcohol. In further embodiments, the compositions according to the disclosure comprise combinations of at least one liquid fatty alcohol and at least one solid fatty alcohol.

According to some embodiments, liquid fatty alcohols that can be used may contain C10-C34, and may have branched carbon chains and/or have branched and/or unsaturated (C═C double bond), and contain from 12 to 40 carbon atoms.

The liquid fatty alcohols may be represented by the formula R—OH, wherein R denotes a C12-C24 branched alkyl group or an alkenyl group (comprising at least one C12-C24 double bond C═C), R being optionally substituted by one or more hydroxy groups. In some embodiments, a liquid fatty alcohol is a branched saturated alcohol. In at least certain embodiments, R does not contain a hydroxyl group. Non-limiting examples of liquid fatty alcohols may include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, and 2-tetradecyl-1-cetanol. In some particular embodiments, the liquid fatty alcohol is 2-octyl-1-dodecanol.

According to further embodiments, solid fatty alcohols that can be used may include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm. The solid fatty alcohols may be soluble, under the same temperature and pressure conditions, in at least one organic solvent (for example ethanol, chloroform, benzene or liquid petroleum jelly) to at least 1% by weight.

The solid fatty alcohols may be represented by the formula R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

Non-limiting examples of solid fatty alcohols include lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol); or mixtures thereof. In some embodiments, the compositions disclosed herein comprise at least one solid fatty alcohol chosen from cetyl alcohol, stearyl alcohol, behenyl alcohol and mixtures thereof such as cetylstearyl or cetearyl alcohol.

Fatty alcohol derivatives that can be used may include methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG-8-ceteth-1, and PPG-10 cetyl ether; and mixtures of all of the foregoing compounds.

In some embodiments, compositions according to the disclosure comprise a fatty alcohol that is hydrogenated (for example, stearyl, lauryl, cetyl, cetearyl). In some embodiments, the fatty alcohol contains one or more double bonds (for example, oleyl). In some embodiments, an emulsifier comprised in the compositions is chosen from decyl alcohol, undecyl alcohol, dodecyl alcohol, myristyl alcohol, lauryl alcohol, cetyl alcohol, stearyl alcohol, cetearyl alcohol (a combination of cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, or a combination thereof. In some embodiments, an emulsifier comprised in the compositions may be chosen from myristyl alcohol, lauryl alcohol, cetyl alcohol, stearyl alcohol, cetearyl alcohol, isostearyl alcohol, oleyl alcohol, isotridecyl alcohol, or a combination thereof.

Thus, without intending to be limiting, the at least one emulsifier may be chosen from C9-C11 alcohols, C12-C13 alcohols, C12-C15 alcohols, C12-C16 alcohols, C14-C15 alcohols, C12-C22 alcohols, arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, 2-octyldodecanol, isostearyl alcohol, 2-hexyldecanol, 2-heptyldecanol, 2-octyldecanol, caproic alcohol (1-hexanol), enanthic alcohol (1-heptanol), caprylic alcohol (1-octanol), pelargonic alcohol (1-nonanol), capric alcohol (1-decanol), lauryl alcohol (1-dodecanol), or mixtures thereof.

In one embodiment, the at least one fatty alcohol is cetyl alcohol. In one embodiment, the at least one fatty alcohol is cetearyl alcohol. In one embodiment, the at least one fatty alcohol comprises both cetyl alcohol and cetearyl alcohol by way of example. In some embodiments, an emulsifier may be chosen from ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, laureth-1, laureth-2, laureth-3, laureth-4, laureth-5, laureth-6, laureth-7, laureth-8, laureth-9, laureth-10, laureth-11, laureth-12, laureth-13, laureth-14, laureth-15, lauretih-16, laureth-20, laureth-23, laureth-25, laureth-30, laureth-40, deceth-3, deceth-5, oleth-5, oleth-30, steareth-2, steareth-10, steareth-20, steareth-100, cetylsteareth-12, ceteareth-5, ceteareth-5, polyglyceryl 4-lauryl ether, polyglyceryl 4-oleyl ether, polyglyceryl 2-oleyl ether, polyglyceryl 2-cetyl ether, polyglyceryl 6-cetyl ether, polyglyceryl 6-oleylcetyl ether, polyglyceryl 6-octadecyl ether, C9-C11 pareth-3, C9-C11 pareth-6, C11-C15 pareth-3, C11-C15 pareth-5, C11-C15 pareth-12, C11-C15 pareth-20, C12-C15 pareth-9, C12-C15 pareth-12, C22-C24 pareth-33, or a mixture thereof.

### Glyceryl Esters (Glycerol Esters)

In some embodiments, compositions according to the disclosure comprise at least one emulsifier chosen from one glyceryl (or glycerol) esters, or derivatives thereof.

The at least one glyceryl ester may have a carbon chain of 8 to 24 carbons, and may be chosen from: i) esters of an oligomeric glycerol; ii) the arachidyl propionate sold under the trade mark WAXENOL 801 by Alzo; iii) phytosterol esters, triglycerides of fatty acids and derivatives thereof, such as hydrogenated cocoglycerides; iv) noncrosslinked polyesters resulting from the poly condensation between a linear or branched C4-C50 dicarboxylic acid or polycarboxylic acid and a C2-C50 diol or polyol; v) aliphatic esters of an ester resulting from the esterification of an aliphatic hydroxycarboxylic acid ester with an aliphatic carboxylic acid; or vi) a mixture thereof.

Non-limiting examples of the esters of an oligomeric glycerol include diglycerol, such as the condensates of adipic acid and of glycerol, for which a portion of the hydroxyl groups of the glycerols have reacted with a mixture of fatty acids, such as steric acid, capric acid, stearic and isostearic acid, and 12-hydroxystearic acid, such as those sold under the trade mark SOFTISAN 649 by the company Cremer Oleo or under the trademark SP SUPERMOL B MBAL-SS-(RB) by the company Croda, such as bis-diglyceryl polyacyladipate-2, or may be bis-diglyceryl polyacyladipate-1.

In certain exemplary embodiments, the glycerol esters may be polyglycerol esters of fatty acids (polyglyceryl esters) having a structure in accordance with the following formula (VIII):

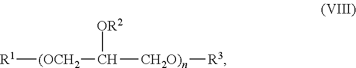

wherein n is from 2 to 20, from 2 to 10, or from 2 to 5, or is 2, 3, 4, 5, 6, 7, 8, 9, or 10, and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀.

Non-limiting examples of nonionic polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, and polyglyceryl-10 stearate.

In some embodiments, the at least one emulsifier comprises at least one glyceryl ester chosen from esters of an oligomeric glycerol, arachidyl propionate, phytosterol esters, triglycerides of fatty acids and derivatives thereof, noncrosslinked polyesters resulting from the poly condensation between a linear or branched C4-C50 dicarboxylic acid or polycarboxylic acid and a C2-C50 diol or polyol, aliphatic esters of an ester resulting from the esterification of an aliphatic hydroxycarboxylic acid ester with an aliphatic carboxylic acid, and a mixture thereof. Non-limiting examples of glyceryl esters include bis-diglyceryl polyacyladipate-1, bis-diglyceryl polyacyladipate-2, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, glyceryl dioleate, glyceryl distearate, glyceryl laurate, or a mixture thereof. In one embodiment, the at least one emulsifier is chosen from glyceryl stearate, bis-diglyceryl polyacyladipate-1, bis-diglyceryl polyacyladipate-2, or a mixture thereof. In another embodiment, at least one emulsifier is chosen from glyceryl esters comprising bis-diglyceryl polyacyladipate-1 and/or bis-diglyceryl polyacyladipate-2, and optionally, a second glyceryl ester. In one other embodiment, the at least one emulsifier comprises a glyceryl ester that is glyceryl stearate, and optionally, a second glyceryl ester. In a preferred embodiment, the composition comprises bis-diglyceryl polyacryladipate-1 and/or bis-diglyceryl polyacryladipate-2, and optionally at least one additional emulsifier.

### Non-Glyceryl Esters

In some embodiments, compositions according to the disclosure may also include an ester other than glyceryl esters (non-glyceryl esters). In some cases, the ester other than glyceryl ester is chosen from isopropyl esters, cetyl esters, or a mixture thereof. Non-limiting examples of isopropyl esters include isopropyl myristate, isopropyl laurate, isopropyl oleate, isopropyl palmitate, and isopropyl stearate. In some embodiments, the ester that is not a glyceryl ester may be chosen from isopropyl myristate, cetyl esters, isopropyl palmitate, or a mixture thereof.

The ester other than glyceryl ester may also be chosen from a fatty ester. Non-limiting examples of fatty esters may include ethoxylated fatty esters, such as the adducts of ethylene oxide with esters of lauric acid, palmitic acid, stearic acid or behenic acid, and mixtures thereof. Exemplary suitable fatty esters may include those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and mixtures thereof.

According to various embodiments, if present, the total amount of the at least one emulsifier chosen from fatty alcohols, esters, and/or derivatives thereof, or the amount of each of the individual fatty alcohols, esters, and/or derivatives thereof, in the composition may range up to about 15%, such from about 0.01% up to about 15% by weight, based on the total weight of the composition. By way of non-limiting example, any of the at least one emulsifier chosen from fatty alcohols, esters, or combinations thereof, may be present in the composition in an amount ranging from about 0.01% to about 10%, about 0.01% to about 9%, about 0.01% to about 8%, about 0.01% to about 7%, about 0.01% to about 6%, about 0.1% to about 10%, about 0.1% to about 9%, about 0.1% to about 8%, about 0.1% to about 7%, or about 0.1% to about 6% by weight, based on the weight of the composition, including ranges and subranges thereof.

In certain embodiment, the at least one emulsifier is present in an amount ranging from about 0.5% to about 8%, such as about 1% to about 7% by weight, based on the weight of the composition.

### Silicones

Compositions according to the disclosure optionally contain at least one silicone compound. In various embodiments, the at least one silicone compound may be chosen from dimethicone, dimethicone copolymers, amino functional silicones, and mixtures thereof.

In some embodiments, the at least one silicone compound may be an amino functional silicone comprising at least one functionalized amodimethicone. The term “amino functional silicone” as used herein can mean any silicone comprising at least one primary, secondary or tertiary amine or a quaternary ammonium group (i.e., a quaternized group).

Non-limiting examples of amino functional silicone that may be used include:

a) polysiloxanes corresponding to formula (A):

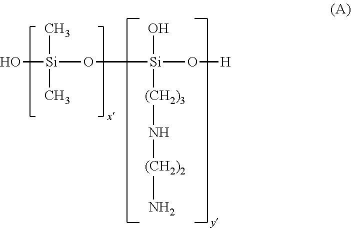

wherein x′ and y′ are integers such that the weight-average molecular weight (Mw) is comprised between about 5000 and 500 000;

b) amino silicones corresponding to formula (B):

R′ₐG₃₋ₐ-Si(OSiG₂)ₙ-(OSiG_{b}R′_{2-b})ₘO—SiG₃₋ₐ-R′ₐ (B)

wherein:
G, which may be identical or different, designate a hydrogen atom, or a phenyl, OH or C₁-C₈ alkyl group, for example methyl, or C₁-C₈ alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10; and
R′, which may be identical or different, denote a monovalent radical having formula —CqH₂qL, in which q is a number ranging from 2 to 8 and L is an optionally quaternized amino group chosen from the following groups:
—NR″-Q-N(R″)₂
—N(R″)₂
—N+(R″)₃A-
—N+H(R″)₂A-
—N+H₂(R″) A-
—N(R″)-Q-N+R″H₂A-
—NR″-Q-N+(R″)₂H A-
—NR″-Q-N+(R″)₃A-,
in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A- represents a cosmetically acceptable ion, in particular a halide such as fluoride, chloride, bromide or iodide.

A group of amino silicones corresponding to formula (B) is represented by the silicones called “trimethylsilylamodimethicone” having formula (C):

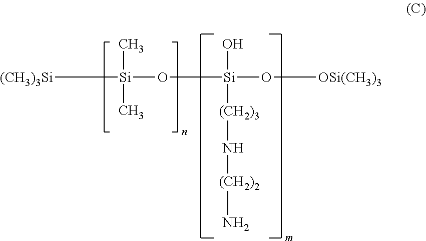

in which n and m have the meanings as in formula B.

Another group of amino silicones corresponding to formula (B) is represented by silicones of formula (D):

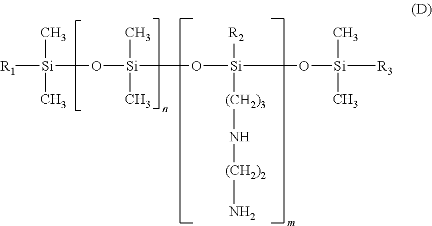

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5; and
R₁, R₂, R₃, which may be identical or different, represent a hydroxy or C₁-C₄ alkoxy radical, where at least one of the radicals R₁ to R₃ denotes an alkoxy radical.

Another group of amino silicones corresponding to formula (B) is represented by silicones of formula (E):

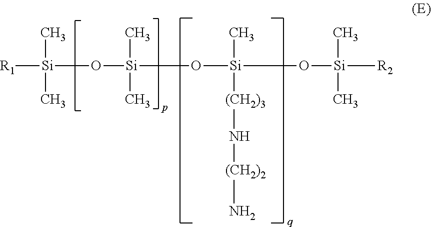

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5; and
R₁, R₂, which are different, represent a hydroxy or C₁-C₄alkoxy radical, where at least one of the radicals R₁ or R₂ denotes an alkoxy radical.

Commercial products corresponding to these silicones having structure (D) or (E) may include in their composition one or more other amino silicones whose structure is different than formulae (D) or (E). For example, a product containing amino silicones having structure (D) is sold by Wacker under the name Belsil® ADM 652, and products containing amino silicones having structure (E) include those sold by Wacker under the names Fluid WR 1300® or Finish CT 96 E® or SLM 28020®.

Another group of amino silicones corresponding to formula (B) is represented by the following formula (F):

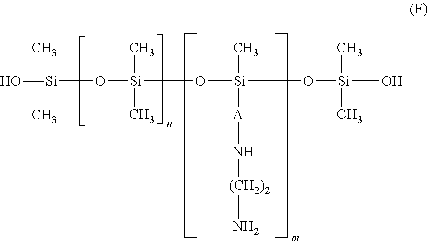

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10; and
A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1,000,000 and even more particularly from 3500 to 200,000. A preferred silicone of formula (F) is amodimethicone (INCI name) sold under the tradename XIAMETER® MEM-8299 Cationic Emulsion by Dow Corning or sold under the tradename SILSOFT 253, by Momentive Performance Materials.

Another group of amino silicones corresponding to formula (B) is represented by the following formula (G):

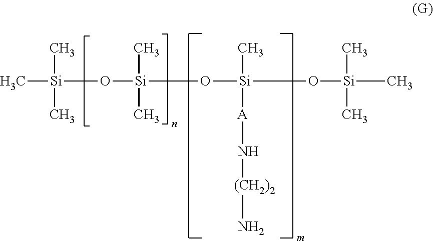

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10; and
A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1,000,000 and even more particularly from 1000 to 200,000. A commercially available silicone having this formula is DC2-8566 Amino Fluid by Dow Corning.

c) amino silicones corresponding to formula (H):

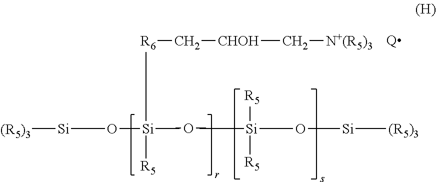

in which:
R5 represents a monovalent hydrocarbon-based radical containing from 1 to 18 carbon atoms, and in particular a C₁-C₁₈ alkyl or C₂-C₁₈ alkenyl radical, for example methyl;
R6 represents a divalent hydrocarbon-based radical, in particular a C₁-C₁₈ alkylene radical or a divalent C₁-C₁₈, for example C₁-C₈, alkylenoxy radical linked to the Si via an SiC bond;
Q⁻ is an anion such as a halide ion, in particular chloride, or an organic acid salt (for example acetate);
r represents a mean statistical value from 2 to 20 and in particular from 2 to 8; and
s represents a mean statistical value from 20 to 200 and in particular from 20 to 50.

Examples of such amino silicones are described in U.S. Pat. No. 4,185,087.

d) quaternary ammonium silicones having formula (I):

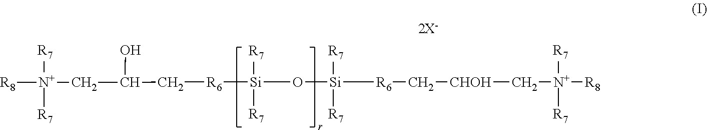

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R6 represents a divalent hydrocarbon-based radical, in particular a C₁-C₁₈ alkylene radical or a divalent C₁-C₁₈, for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;
X⁻ is an anion such as a halide ion, in particular chloride, or an organic acid salt (for example acetate); and
r represents a mean statistical value from 2 to 200 and in particular from 5 to 100.

Examples of such silicones are described, for example, in patent application EP-A 0 530 974.

e) amino silicones having formula (J):

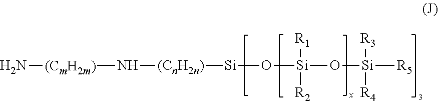

in which:
R₁, R₂, R₃ and R₄, which may be identical or different, denote a C₁-C₄ alkyl radical or a phenyl group;
R₅ denotes a C₁-C₄ alkyl radical or a hydroxyl group;
n is an integer ranging from 1 to 5;
m is an integer ranging from 1 to 5; and
x is chosen such that the amine number is between 0.01 and 1 meq/g.

f) multiblock polyoxyalkylenated amino silicones, of type (AB)n, A being a polysiloxane block and B being a polyoxyalkylenated block containing at least one amine group. In various embodiments, such silicones may comprise repeating units having one of the following general formulae:

[—(SiMe₂O)xSiMe₂-R—N(R″)—R′—O(C₂H₄₀)ₐ(C₃H₆O)b-R′—N(H)—R—]

[—(SiMe₂O)xSiMe₂-R—N(R″)—R′—O(C₂H₄O)ₐ(C₃H₆O)b- ]

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical; and
R′, which may be identical or different, represent a divalent linear or branched C₂-C₁₂ hydrocarbon-based radical, optionally including one or more heteroatoms such as oxygen; preferably, R′ denotes an ethylene radical, a linear or branched propylene radical, a linear or branched butylene radical, or a —CH₂CH₂CH₂OCH(OH)CH₂— radical; preferentially R′ denotes —CH(CH₃)—CH₂—.

The siloxane blocks preferably represent from 50-95 mol % of the total weight of the silicone, more particularly from 70-85 mol %. The amine content is preferably from 0.02 to 0.5 meq/g of copolymer in a 30% solution in dipropylene glycol, more particularly from 0.05 to 0.2. The weight-average molecular weight (Mw) of the silicone is preferably comprised between 5000 and 1,000,000, more particularly between 10,000 and 200,000. Non-limiting examples include bis-amino PEG/PPG-41/3 aminoethyl PG-propyl dimethicone and PEG-40/PPG-8 methylaminopropyl hydroxypropyl dimethicone copolymer. Commercially available products include the silicones sold under the names SILSOFT A-843 or SILSOFT A+ by Momentive.

g) the alkylamino silicones corresponding to formula (K) below:

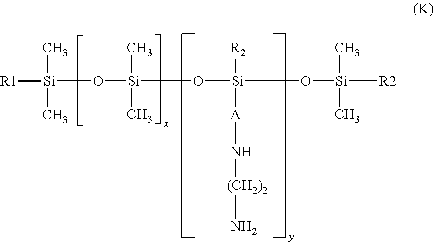

in which:
x and y are numbers ranging from 1 to 5000; preferably, x ranges from 10 to 2000 and especially from 100 to 1000; preferably, y ranges from 1 to 100;
R₁ and R₂, which may be identical or different, preferably identical, are linear or branched, saturated or unsaturated alkyl radicals, comprising 6 to 30 carbon atoms, preferably 8 to 24 carbon atoms and especially 12 to 20 carbon atoms; and
A denotes a linear or branched alkylene radical containing from 2 to 8 carbon atoms.

In various exemplary embodiments, A comprises from 3 to 6 carbon atoms, especially 4 carbon atoms, and in certain embodiments, A is branched. Mention may be made of the following divalent radicals: —CH₂CH₂CH₂ and —CH₂CH(CH₃)CH₂—.

Preferably, R₁ and R₂, which may be identical or different, are saturated linear alkyl radicals comprising 6 to 30 carbon atoms, preferably 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R₁ and R₂, which may be identical or different, are chosen from hexadecyl (cetyl) and octadecyl (stearyl) radicals.

In various exemplary embodiments, the silicone of formula (K) is chosen such that x ranges from 10 to 2000 and especially from 100 to 1000; y ranges from 1 to 100; A comprises 3 to 6 carbon atoms and especially 4 carbon atoms; preferably, A is branched; and more particularly A is chosen from the following divalent radicals: CH₂CH₂CH₂ and —CH₂CH(CH₃)CH₂—; and R₁ and R₂, which may be identical or different, are linear, saturated alkyl radicals comprising 6 to 30 carbon atoms, preferably 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R₁ and R₂, which may be identical or different, being chosen from hexadecyl (cetyl) and octadecyl (stearyl) radicals.

An exemplary amino silicone of formula (K) is bis-cetearylamodimethicone (INCI name), such as the silicone sold under the name SILSOFT AX by Momentive.

h) silicone compounds with at least one quaternary ammonium group. Suitable non-limiting examples are quaternium 80, silicone quaternium-1, silicone quaternium-2, silicone quaternium-2 panthenol succinate, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-16, silicone quaternium-16/Glycidoxy Dimethicone Crosspolymer, silicone quaternium-17, silicone quaternium-18, silicone quaternium-20 and silicone quaternium-21. Preferred are quaternium 80, silicone quaternium-16, silicone quaternium-18, silicone quaternium-1, silicone quaternium-2, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-17, silicone quaternium-20 and silicone quaternium-21. More preferred are quaternium 80, silicone quaternium-16, silicone quaternium-18, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, and silicone quaternium-17. For example, quaternium 80, silicone quaternium-16, silicone quaternium-18, silicone quaternium-15, or mixtures thereof may be chosen.

The amount of the at least one silicone compound that may be included in various embodiments can vary but, if present, typically ranges from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 1.5% to about 2%, from about 2% to about 10%, from about 2% to about 9%, from about 2% to about 8%, from about 2% to about 7%, from about 2% to about 6%, from about 2% to about 5%, from about 2% to about 4%, or from about 2% to about 3% by weight, relative to the total weight of the composition.

### Oils

Compositions according to the disclosure optionally comprise at least one oil, which may, in some embodiments, be a low molecular weight oil. The oil may, in certain embodiments, be chosen from oils which are derived from a plant, for example, oils from seeds or fruits.

Oils that may be used according to the disclosure include, by way of example, a hydrocarbon-based oil of plant origin and/or vegetable origin. Non-limiting examples of such oils include acai oil, almond oil, aloe vera oil, andiroba oil, annatto oil, avocado oil, babassu oil, borage oil, brazil nut oil, buriti oil, camelina oil, coffee oil, copaiba oil, emu oil, passion fruit oil, almond oil, castor oil, coconut oil, grapeseed oil, jojoba oil, macadamia nut oil, rose hip oil, ajwain oil, angelic root oil, anise oil, aragan oil, asafetida, balsam oil, basil oil, bay oil, bergamot oil, black pepper essential oil, buchu oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, cypress, cypriol, curry leaf, davana oil, dill oil, elecampane, eucalyptus oil, fennel seed oil, fenugreek oil, fir, frankincense oil, galangal, geranium oil, ginger oil, goldenrod, grapefruit oil, grapeseed oil, henna oil, helichrysum, horseradish oil, hyssop, Idaho tansy, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, patchouli oil, perilla oil, pennyroyal oil, peppermint oil, petitgrain, pine oil, plum oil, ravensara, red cedar, roman chamomile, rose oil, rosehip oil, rosemary oil, rosewood oil, sandalwood oil, sassafras oil, savory oil, schisandra oil, spikenard, spruce, star anise oil, tangerine, tarragon oil, tea tree oil, thyme oil, tsuga oil, turmeric, valerian, vetiver oil, western red cedar, wintergreen, yarrow oil, ylang-ylang, and zedoary oil.

In some embodiments, oils that may be used according to the disclosure may include an oil chosen from glyceride triesters, which are generally triesters of fatty acids and of glycerol, the fatty acids of which can have varied chain lengths from C₄ to C₂₄, it being possible for these chains to be saturated or unsaturated and linear or branched. Non-limiting examples of these oils include wheat germ oil, sunflower oil, grape seed oil, sesame oil, corn oil, apricot oil, castor oil, shea oil, avocado oil, olive oil, soybean oil, sweet almond oil, palm oil (which contains palmitic acid), rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.

In some embodiments, the oil component in compositions according to the disclosure comprises, consists essentially of, or consists of low molecular weight oils having less than 16 carbons, such as less than 14 carbons, or less than 12 carbons, which may be saturated or unsaturated. In at least some embodiments, the oil component in compositions according to the disclosure comprises, consists essentially of, or consists of coconut oil, shea butter, castor oil, palmitic acid, jojoba oil, or a mixture of two or more of the foregoing.

The total amount of the at least one oil that may be included in various embodiments, if present, ranges from about 0.01% to about 15%, such as from about 0.1% to about 10%, from about 0.1% to about 9%, from about 0.1% to about 8%, from about 0.1% to about 7%, from about 0.1% to about 6%, from about 0.1% to about 5%, from about 0.5% to about 10%, from about 0.5% to about 9%, from about 0.5% to about 8%, from about 0.5% to about 7%, from about 0.5% to about 6%, from about 0.5% to about 5%, from about 1% to about 10%, from about 1% to about 9%, from about 1% to about 8%, from about 1% to about 7%, from about 1% to about 6%, from about 1% to about 5%, from about 2% to about 10%, from about 2% to about 9%, from about 2% to about 8%, from about 2% to about 7%, from about 2% to about 6%, or from about 2% to about 5% by weight, based on the total weight of the composition, including all ranges and subranges therebetween.

In some embodiments, the amount of the at least one oil may be chosen such that it is present in a particular weight ratio relative to the at least one cyclodextrin. By choosing the type and also the amount of the at least one oil relative to the at least one cyclodextrin, it is surprisingly possible to tailor the composition to provide hair elongation benefits and at the same time cosmetic benefits such as softness, smoothness, shine, and/or frizz control, while at the same time minimizing or eliminating the presence of flaking on the hair. For example, the ratio of cyclodextrin:oil may range from 1.2:0.5 to about 1:2, such as from about 1:0.5 to about 1:1.8, or from about 1:0.75 to about 1:1.5, or about 1:0.2 to about 1:0.5, including all ranges and subranges thereof. For example, the ratio of cyclodextrin:oil may be about 1:0.75 to about 1:1.75, about 1:1.25 to about 1:1.5, or about 1:0.75 to about 1:1.5, including all ranges and subranges thereof.

In one embodiment, the at least one oil comprises, consists essentially of, or consists of jojoba oil, and is present in the composition ata ratio of cyclodextrin:oil ranging from about 1:0.75 to about 1:1.75, such as about 1:1.25 to about 1:1.5. In a further embodiment, the at least one oil comprises, consists essentially of, or consists of coconut oil, and is present in the composition at a ratio of cyclodextrin:oil ranging from about 1:0.75 to about 1:1.75, such as about 1:0.75 to about 1:1.5.

### Additional Components

Compositions according to the disclosure may optionally comprise any additional or auxiliary component suitable for use in such compositions. Such components may include, but are not limited to, conditioning agents, dyes/pigments, moisturizing agents, fillers, structuring agents, shine agents, antioxidants, penetrants, sequestrants, fragrances, buffers, dispersants, plant extracts, preserving agents (preservatives), opacifiers, sunscreen agents, pH adjusting agents, vitamins, and antistatic agents.

Optional additional auxiliary components may be present in an amount ranging up to about 15%, such as from about 0.01% to about 15%, about 0.1% to about 10%, or about 0.1% to about 5% by weight, relative to the total weight of the composition.

In various embodiments, the compositions may have a pH less than or equal to about 7, such as about 3 to about 7, about 4 to about 7, or about 5 to about 7. In various embodiments, the pH of the compositions may range from about 2 to about 7, such as from about 3 to about 6, or from about 3.5 to about 5.5, including all ranges and subranges therebetween.

Compositions according to the disclosure are typically in the form of a liquid, cream, paste, foam, or lotion, but are not limited to these forms. In some embodiments, the compositions may be in a bottle, tube, or pump-style bottle. In some embodiments, the compositions are non-aerosol type product.

### II. Methods

The present disclosure also relates to methods for treating the hair, e.g. styling or shaping hair or altering the shape and/or style of hair, for example by improving elongation of curly or wavy hair by the use of the compositions described herein. The methods generally comprise applying any of the compositions according to the disclosure to the hair.

Compositions according to the disclosure are typically used for providing a visible elongation benefit to curly hair, or for improving the elongation of hair curls. The compositions may be useful in a variety of settings, either for chemically treated or untreated hair, and/or for natural or unnatural curls. The degree of curliness or curl type of the hair may vary and is not limited. For example, the curls of hair been treated by the compositions disclosed herein may range from slightly wavy to very kinky and coily hair, and may have different textures and colors.

In addition to improving elongation of hair curls, the compositions may also be used to provide a variety of desirable sensory benefits, for example, moisture, smoothness, softness, good bounce, good shine, and/or anti-frizz, to the hair. As such, the compositions are useful in styling (which includes shaping) hair while also caring for hair, conditioning hair, and/or imparting one or more above described sensory benefits to the hair.

Typically, methods according to the disclosure may include applying an effective amount of a composition disclosed herein to hair having waves or curls, when the hair is dry, wet, damp, or moist. The hair may be either natural or synthetic hair. As used herein, the term “effective amount” refers to an amount sufficient to provide a desired elongation effect to the hair, taking into account the degree of curliness, the length, the volume, and the texture of the hair. In general, from about 0.5 grams to about 50 grams of product is applied to the hair, depending on the specific product formulation, hair length, hair volume, and hair style type. The composition applied to the hair may be distributed evenly by combing through with fingers or a means such as a comb or the like. The composition may then be dried, for example air-dried. The composition may be allowed to remain on the hair as a leave-in product for any period of time as needed, for example, a few hours or a few days, or until the next washing or rinsing of the hair. Alternatively, the compositions may be rinsed from the hair after a brief leave-in period, from about a few seconds (e.g. 5, 10, 20, 30, or 60 seconds) to about 10, 20, or 30 minutes, or longer.

In some embodiments, before applying the composition to the hair, the hair may be first cleansed with a commercially available shampoo, or rinsed with water. The composition is then applied to the washed or rinsed hair when the hair is wet, damp, or moist. In some other embodiments, before applying the composition to dry hair, the hair can optionally be moistened, damped, or wetted by water spray or using a wet towel, or by applying other treatment compositions that make the hair moist, damp, or wet.

In some embodiments, after application of the compositions disclosed herein, the hair may be shaped or styled as needed, such as be twisted or the like. In some embodiments, the hair treated with the compositions may be twisted within 30 minutes, or within 20 minutes, while the hair is wet or damp.

Methods according to the disclosure generally do not require the use of a reducing agent, including a base, or heating the hair for stretching the hair curls or elongating the hair curls. As such, in some embodiments, the method does not including using a reducing agent, or a base, and/or does not including heating the hair.

In various embodiments, the methods of treating hair with the compositions according to the disclosure, impart various long-lasting benefits described above to the hair, without a greasy feel or flaking, relative to hair not having been treated with a composition according to the disclosure. The term “long-lasting,” as used herein, means that the elongation effect and other benefits imparted to the hair may remain a desired length of time, such as a few hours, a few days, or until the hair is rinsed or washed.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the terms “applying a composition onto keratin fibers” and “applying a composition onto hair” and variations of these phrases are intended to mean contacting the keratin materials including hair and skin, with at least one of the compositions of the disclosure, in any manner. It may also mean contacting the keratin materials in an effective amount.

Unless otherwise indicated, all percentages herein are by weight, relative to the weight of the total composition.

As used herein, the term “styling” is intended to include “shaping.”

As used herein, the term “curly hair” refers to any hair including a curl. The curl may be natural or unnatural, i.e., formed by chemical treatment or physical treatment of the hair. The degree of curliness of the hair may vary and is not limited.

As used herein, hair with improved or enhanced curl definition may have curls with a shape that has a clean ringlet appearance rather than being frizzy, curls that appear more individualized, curls that are more closed in appearance, and/or curls that have an improved visual appearance of the hair color and/or highlights.

As used herein, the terms “elongating” and “stretching” curly hair are interchangeable, and both refer to reducing the degree of curliness and/or tightness of curls of the hair, and “improving the elongation of hair curls” and variants thereof means that the length of the hair appears closer to the actual, uncurled length of the hair.

A “leave-in” composition or product refers to a composition such as a hair-treatment composition that is not rinsed and/or washed away with water or acceptable solvent after the application of the composition onto the keratin fiber, such as hair; instead, the composition is allowed to remain on the keratin fibers for a period of time as desired, such from 1 hour, 2 hours, 3 hours, 4 hours, up to 8 hours, overnight, or as long as needed, until next time of washing or rinsing the keratin fibers.

As used herein, the term “polyol” refers to an organic molecule comprising at least two free hydroxyl groups.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “salts” refers to throughout the disclosure may include salts having a counter-ion such as an alkali metal, alkaline earth metal, or ammonium counterion. This list of counterions, however, is non-limiting.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “surfactants,” as well as any specifically-identified surfactants, includes salts of the surfactants even if not explicitly stated.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt%) of active materials, relative to the total weight of the composition, unless otherwise indicated.

### Example 1—Demonstration of Elongation Benefits to Hair

The comparative and inventive compositions set forth in Table 1-1 below were prepared.

TABLE 1-1
Compositions

| | Com- | | | | | | |
|---|---|---|---|---|---|---|---|
| | parative | | Inventive | | | | |
| | C1 | C2 | 1A | 1B | 1C | 1D | 1E |
| β-CYCLODEXTRIN | — | — | 2 | 4 | 6 | 8 | 10 |
| HEXYLENE | — | 2.5 | 0.5 | 1 | 1.5 | 2 | 2.5 |
| GLYCOL | | | | | | | |
| WATER | QS to | QS to | QS to | QS to | QS to | QS to | QS to |
| | 100 | 100 | 100 | 100 | 100 | 100 | 100 |

Each of compositions C1 and 1A-1E were tested on three swatches of curly hair, and composition C2 was tested on two swatches of curly hair. First, the swatches were washed with a standard shampoo and then one of compositions C1-C2 or 1A-1E was applied and the length of each swatch was immediately recorded (T0). After 24 hours at ambient temperature and humidity, the length of the swatches was again recorded (T24H). The difference in the lengths of hair is recorded in Table 1-2.

TABLE 1-2
Difference in Length of Treated Swatches After 24 Hours (Inches)

| | C1 | C2 | 1A | 1B | 1C | 1D | 1E |
|---|---|---|---|---|---|---|---|
| T0 | 6.84 | 7.09 | 7.07 | 7.14 | 7.16 | 7.13 | 7.15 |
| T24H | 5.66 | 5.81 | 6.18 | 6.70 | 6.78 | 6.73 | 6.75 |
| Difference | 1.19 | 1.28 | 0.89 | 0.44 | 0.38 | 0.39 | 0.40 |

As can be seen in Table 1-2, the swatches treated with compositions C1 and C2 showed the least elongation over a period of 24 hours. However, swatches treated with each of the inventive compositions 1A-1E demonstrated curl elongation over the same period, with compositions 1B-1E all having approximately the same result.

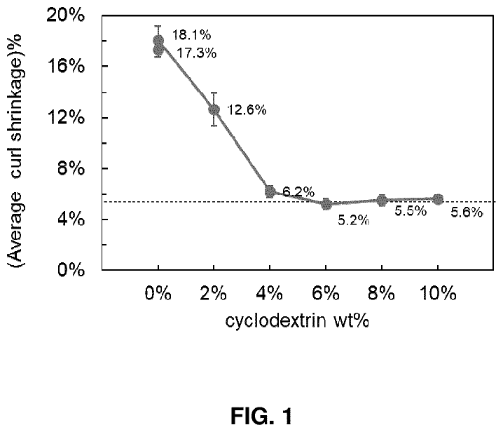

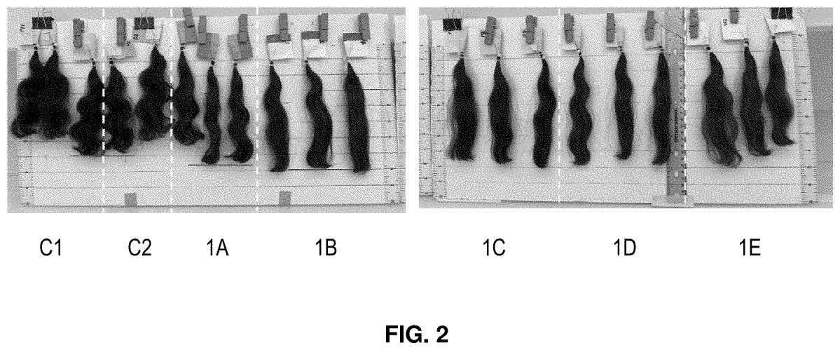

FIG. 1 is a graph demonstrating the results in Table 1-2 at T24H, and FIG. 2 shows images of the treated swatches at T24H. This example confirms that the use of cyclodextrins provides a curl-elongation benefit to hair.

### Example 2—Demonstration of Benefits to Hair

The comparative and inventive compositions set forth in Table 2 below were prepared.

TABLE 2
Compositions

| | Inventive | | | | Comparative | |
|---|---|---|---|---|---|---|
| INCI NAME | 2A | 2B | 2C | 2D | C3 | C4 |
| CETYL ESTERS | 1.0 | 1.0 | 1.0 | 1.0 | 1.0 | 0.25 |
| TRIDECETH-6 | 0.12 | 0.12 | 0.12 | 0.12 | 0.12 | |
| PYRIDOXINE HCl | | | | 0.05 | 0.05 | |
| ISOPROPYL ALCOHOL | 0.18 | 0.18 | 0.18 | 0.18 | 0.18 | 0.36 |
| NIACINAMIDE | | | | 0.05 | 0.05 | |
| BEHENTRIMONIUM | 0.79 | 0.79 | 0.79 | 0.79 | 0.79 | 1.58 |
| CHLORIDE | | | | | | |
| PYRUS MALUS (APPLE) | | | | 0.25 | 0.25 | |
| FRUIT EXTRACT | | | | | | |
| ARGANIA SPINOSA | | | | 0.10 | 0.10 | |
| KERNEL OIL | | | | | | |
| CETRIMONIUM | 0.02 | 0.02 | 0.02 | 0.02 | 0.02 | |
| CHLORIDE | | | | | | |
| CAPRYLYL GLYCOL | | | | <0.01 | <0.01 | 0.3 |
| HEXYLENE GLYCOL | 1.15 | 1.15 | 1.15 | | | |
| CHLORHEXIDINE | 0.03 | 0.03 | 0.03 | 0.03 | 0.03 | |
| DIHYDROCHLORIDE | | | | | | |
| TOCOPHEROL | | | | 0.01 | 0.01 | |
| CETEARYL ALCOHOL | 5.00 | 5.00 | 5.00 | 5.00 | 5.00 | 3.0 |
| AMODIMETHICONE | 1.39 | 1.39 | 1.39 | 1.39 | 1.39 | |
| SACCHARUM | | | | 0.02 | 0.02 | |
| OFFICINARUM | | | | | | |
| (SUGARCANE) | | | | | | |
| EXTRACT | | | | | | |
| HYDROGENATED | | | | | | 7.0 |
| STARCH | | | | | | |
| HYDROLYSATE | | | | | | |
| HYDROXYPROPYL | | | | | | 1.25 |
| GUAR | | | | | | |
| GLYCERYL STEARATESE | | | | | | 1.0 |
| GLYCERIN | | | | | | 5.0 |
| BIS-DIGLYCERYL | | | | | | 1.0 |
| POLYACYLADIPATE-2 | | | | | | |
| BUTYROSPERMUM | | | | | | 2.0 |
| PARKII (SHEA) BUTTER | | | | | | |
| RICINUS COMMUNIS | | | | | | 2.0 |
| (CASTOR) SEED OIL | | | | | | |
| β-CYCLODEXTRIN | 3.96 | 3.96 | 3.96 | | | |
| COCONUT OIL | 4.60 | | | | | |
| JOJOBA OIL | | 4.60 | | | | |
| ADDITIVES (pH | | | | 0.74 | 0.74 | 1.6 |
| adjusters, preservatives, | | | | | | |
| dyes, fragrances, plant | | | | | | |
| extracts, humectants) | | | | | | |
| WATER | QS to | QS to | QS to | QS to | QS to | QS to |
| | 100 | 100 | 100 | 100 | 100 | 100 |
| β-CYCLODEXTRIN | | | | 10* | | |
| PENTYLENE GLYCOL | | | | 7* | | |

*These components were added as a super-addition to composition 2D, i.e. the ingredients listed above already added to 100%, before the cyclodextrin and pentylene glycol were added.

In order to demonstrate the beneficial effects on hair, including curl elongation as well as softness, smoothness, shine, and/or frizz control, two half-head studies were conducted, with compositions 2D and C3-C4. First, the hair was washed with a standard shampoo, and then composition 2D was applied to one-half of each mannequin head, and either composition C3 or C4 was applied to the other half of the heads.

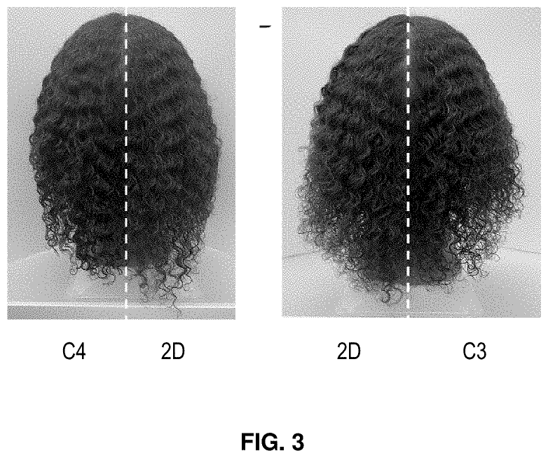

After a period of 16 hours, the hair was evaluated. The hair on both sides of each mannequin head was soft, smooth, shiny, and had excellent frizz control. However, in both comparisons, the half of the head treated with composition 2D was noticeably longer than the half of the head treated with either composition C3 or C4. FIG. 3 shows images of the comparisons, where the difference in length of the hair is noticeable.

The results in this example demonstrate that compositions according to the disclosure comprising deliver improvement in elongation of curls, while at the same time imparting sensory benefits such as smoothness, shine, softness, and frizz control to the hair.

### Example 3—Demonstration of Benefits Across Range of Cyclodextrins Over Time

A study was done to demonstrate that the beneficial effects on hair of compositions according to the disclosure are seen across a range of concentrations of cyclodextrins, which lasts over time.

TABLE 3
Compositions

| | Inventive | | | | | | Comp. |
|---|---|---|---|---|---|---|---|
| INCI NAME | 3A | 3B | 3C | | 3D | 3E | 3F |
| CETYL ESTERS | 1.0 | | | | | | |
| TRIDECETH-6 | 0.12 | | | | | | |
| ISOPROPYL | 0.18 | | | | | | |
| ALCOHOL | | | | | | | |
| BEHENTRIMONIUM | 0.79 | | | | | | |
| CHLORIDE | | | | | | | |
| CETRIMONIUM | 0.02 | | | | | | |
| CHLORIDE | | | | | | | |
| CHLORHEXIDINE | 0.03 | | | | | | |
| DIHYDROCHLORIDE | | | | | | | |
| HEXYLENE GLYCOL | 1.15 | | | | | | |
| CETEARYL | 5.00 | | | | | | |
| ALCOHOL | | | | | | | |
| AMODIMETHICONE | 1.39 | | | | | | |
| β-CYCLODEXTRIN | 1 | 2 | 3 | 4 | 5 | 6 | 0 |
| WATER | QS to | QS to | QS to | QS to | QS to | QS to | QS to |
| | 100 | 100 | 100 | 100 | 100 | 100 | 100 |

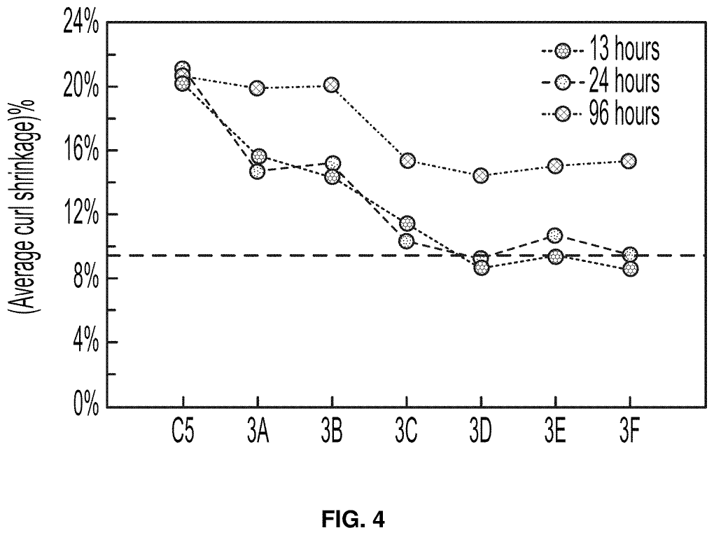

For this study, compositions 3A-3F and C5 were applied to freshly washed swatches of curly hair. The swatches were patted to remove excess composition, brushed with a comb ten times, and left to air dry under ambient conditions. Curl shrinkage was measured after 13 hours, 24 hours, and 96 hours. The results are seen in the graph in FIG. 4. As shown in FIG. 4, at all time points measured, each of compositions 3A-3F provided superior curl shrinkage benefits compared to that of composition C5.

This example demonstrates that a range of concentrations of cyclodextrin provides excellent curl-elongation properties to hair, which properties last over time (i.e. at least 96 hours).

### Example 4 — Demonstration of Benefits of Cyclodextrins With Different Oils

A study was done to demonstrate the benefits of curl elongation as well as softness, smoothness, shine, and/or frizz control could be achieved using a range of different oils in combination with cyclodextrin.

TABLE 4
Compositions

| INCI NAME | C6 | 4A | 4B | 4C | 4D | 4E | 4F |
|---|---|---|---|---|---|---|---|
| CETYL ESTERS | 1.0 | | | | | | |
| TRIDECETH-6 | 0.12 | | | | | | |
| PYRIDOXINE HCl | 0.05 | | | | | | |
| ISOPROPYL | 0.18 | | | | | | |
| ALCOHOL | | | | | | | |
| NIACINAMIDE | 0.05 | | | | | | |
| BEHENTRIMONIUM | 0.79 | | | | | | |
| CHLORIDE | | | | | | | |
| PYRUS MALUS | 0.25 | | | | | | |
| (APPLE) FRUIT | | | | | | | |
| EXTRACT | | | | | | | |
| ARGANIA SPINOSA | 0.10 | | | | | | |
| KERNEL OIL | | | | | | | |
| CETRIMONIUM | 0.02 | | | | | | |
| CHLORIDE | | | | | | | |
| CAPRYLYL GLYCOL | <0.01 | | | | | | |
| CHLORHEXIDINE | 0.03 | | | | | | |
| DIHYDROCHLORIDE | | | | | | | |
| CETEARYL | 5.00 | | | | | | |
| ALCOHOL | | | | | | | |
| AMODIMETHICONE | 1.39 | | | | | | |
| SACCHARUM | 0.02 | | | | | | |
| OFFICINARUM | | | | | | | |
| (SUGARCANE) | | | | | | | |
| EXTRACT | | | | | | | |
| ADDITIVES (pH | 0.75 | | | | | | |
| adjusters, | | | | | | | |
| preservatives, dyes, | | | | | | | |
| fragrances, plant | | | | | | | |
| extracts, humectants) | | | | | | | |
| WATER | QS to 100 | | | | | | |
| β-CYCLODEXTRIN | | 10* | 10* | 10* | 10* | 10* | 10* |
| COCONUT OIL | | | 10* | | | | |
| BUTYROSPERMUM | | | | 10* | | | |
| PARKII (SHEA) | | | | | | | |
| BUTTER | | | | | | | |
| RICINUS COMMUNIS | | | | | 10* | | |
| (CASTOR) SEED OIL | | | | | | | |
| PALMITIC ACID | | | | | | 10* | |
| JOJOBA OIL | | | | | | | 10* |

*These components were added as a super-addition to compositions 4A-4F, i.e. the ingredients listed above already added to 100%, before the cyclodextrin and/or oils were added.

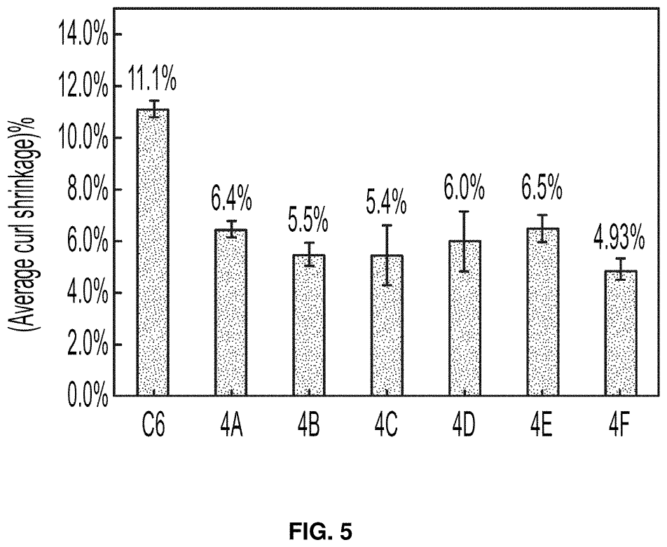

Each of compositions 4A-4F and C6 were applied to freshly washed swatches of curly hair. The swatches were patted to remove excess composition, brushed with a comb ten times, and left to air dry under ambient conditions. Curl shrinkage was measured after 24 hours. The results are seen in the graph in FIG. 5. As shown in FIG. 5, each of compositions 4A-4F provided superior curl shrinkage compared to that of composition C6. Notably, the hair treated with each of compositions 4A-4F and C6 was soft, smooth, shiny, and had excellent frizz control.

This example demonstrates that the beneficial effects imparted to hair by compositions according to the disclosure can be achieved with a range of different combinations of cyclodextrin and oils.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1. A composition for treating keratin fibers, comprising:
(a) at least one cyclodextrin or derivative thereof;
(b) at least one solvent;
(c) at least one cationic surfactant;
(d) at least one emulsifier;
(e) at least one silicone; and
(f) at least one oil.

2. The composition of claim 1, wherein the at least one cyclodextrin or derivative thereof is chosen from those of formula: 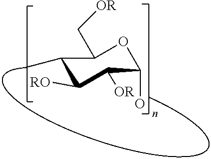
wherein:
R is chosen from H, CH₃, or a hydroxypropyl group, and
n ranges from 6-8.

3. The composition of claim 1, wherein the at least one cyclodextrin or derivative thereof is chosen from α-cyclodextrin, β-cyclodextrin, γ-cyclodextrin, methyl derivatives of α-cyclodextrin, β-cyclodextrin, or γ-cyclodextrin, hydroxypropyl derivatives of α-cyclodextrin, β-cyclodextrin, or γ-cyclodextrin, or mixtures thereof.

4. The composition of claim 1, wherein the at least one cyclodextrin or derivative thereof is present in an amount ranging from about 0.01% to about 20% by weight, relative to the weight of the composition.

5. The composition of claim 1, wherein the at least one solvent comprises water and optionally at least one additional solvent that can improve the solubility of the at least one cyclodextrin.

6. The composition of claim 5, wherein the solvent comprises at least one additional solvent that can improve the solubility of the at least one cyclodextrin chosen from linear or branched, saturated or unsaturated, and substituted or unsubstituted C₃-C₈ polyols.

7. The composition of claim 6, wherein the weight ratio of cyclodextrin:additional solvent ranges from about 1.2:0.1 to about 1:1.

8. The composition of claim 7, wherein the at least one additional solvent is chosen from hexylene glycol, pentylene glycol, or a mixture thereof.

9. The composition of claim 1, wherein the at least one oil is chosen from saturated or unsaturated plant oils having less than 16 carbon atoms.

10. The composition of claim 1, wherein the weight ratio of cyclodextrin:oil ranges from about 1.2:0.5 to about 1:2.

11. The composition of claim 10, wherein the at least one oil is chosen from coconut oil, shea butter, castor oil, palmitic acid, jojoba oil, or mixtures thereof.

12. A composition for treating keratin fibers, comprising:
(a) at least one cyclodextrin or derivative thereof;
(b) at least one solvent;
(c) optionally at least one cationic surfactant;
(d) optionally at least one emulsifier;
(e) optionally at least one silicone; and
(f) optionally at least one oil;
wherein the at least one solvent comprises water and at least one additional solvent that can improve the solubility of the at least one cyclodextrin; and
wherein the weight ratio of cyclodextrin:additional solvent ranges from about 1.2:0.1 to about 1:1.

13. The composition of claim 12, wherein the at least one additional solvent is chosen from linear or branched, saturated or unsaturated, and substituted or unsubstituted C₃-C₈ polyols.

14. The composition of claim 12, wherein the at least one additional solvent is chosen from hexylene glycol, pentylene glycol, or a mixture thereof.

15. The composition of claim 12, wherein the at least one cyclodextrin is chosen from those of formula: 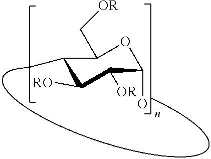
wherein:
R is chosen from H, CH₃, or a hydroxypropyl group, and
n ranges from 6-8.

16. The composition of claim 12, wherein the at least one cyclodextrin or derivative thereof is chosen from α-cyclodextrin, β-cyclodextrin, γ-cyclodextrin, methyl derivatives of α-cyclodextrin, β-cyclodextrin, or γ-cyclodextrin, hydroxypropyl derivatives of α-cyclodextrin, β-cyclodextrin, or γ-cyclodextrin, or mixtures thereof.

17. The composition of claim 12, comprising at least one oil chosen from saturated or unsaturated plant oils having less than 16 carbon atoms.

18. The composition according to claim 17, wherein the weight ratio of cyclodextrin:oil ranges from about 1.2:0.5 to about 1:2.

19. A method for treating hair comprising applying to the hair a composition according to claim 12.

20. A method for treating hair comprising applying to the hair a composition comprising:
(a) at least one cyclodextrin or derivative thereof;
(b) at least one solvent;
(c) at least one cationic surfactant;
(d) at least one emulsifier;
(e) at least one silicone; and
(f) at least one oil.
